(19) United States
(12) Patent Application Publication
(10) Pub. No.: US 2009/0124962 A1
(43) Pub. Date: May 14, 2009

(54) Reflux control in microsurgical system

(76) Inventors: Mark A. Hopkins, Mission Viejo, CA (US); Shawn X. Gao, Irvine, CA (US)

Correspondence Address: ALCON, IP LEGAL, TB4-8, 6201 SOUTH FREEWAY, FORT WORTH, TX, 76134, US

(21) Appl. No.: 12/355,276

(22) Filed: Jan. 16, 2009

Related U.S. Application Data
(63) Continuation of application No. 11/474,190, filed on Jun. 23, 2006.

(51) Int. Cl. A61F 9/007

(52) U.S. Cl. 604/24; 604/28

(57) ABSTRACT

A microsurgical system capable of improving reflux via programmable, pre-defined reflux profiles and a pressure accumulator.

This application is a continuation of U.S. application Ser. No. 11/474,190 filed Jun. 23, 2006.

### FIELD OF THE INVENTION

The present invention generally pertains to controlling reflux in microsurgical systems and more particularly to controlling reflux in ophthalmic microsurgical systems.

### DESCRIPTION OF THE RELATED ART

During small incision surgery, and particularly during ophthalmic surgery, small probes are inserted into the operative site to cut, remove, or otherwise manipulate tissue. During these surgical procedures, fluid is typically infused into the eye, and the infusion fluid and tissue are aspirated from the surgical site. These probes have small orifices that are easily clogged with tissue. Such clogging is typically referred to as “occlusion”, “tip occlusion”, or “port occlusion”. The process of clearing such occlusions is typically referred to as “reflux”.

More generally, reflux is the ability to reverse the direction of the aspiration flow in a surgical system. Reflux may also be used for visualization of the surgical site (e.g. by moving blood and other tissue away from a particular point of interest).

A traditional method of reflux is to create a backpressure pulse of fluid that travels through the aspiration circuit to the tip or port of the probe to clear the incarcerated tissue. A hammer or valve is used to pinch a silicone tube to create a positive pressure pulse. This approach has no ability to control the reflux pressure profile. Therefore, a need continues to exist for an improved method of controlling reflux in a microsurgical system.

### SUMMARY OF THE INVENTION

The present invention provides improved apparatus and methods for controlling reflux in a microsurgical system. In one aspect of the present invention, an apparatus for controlling reflux in a microsurgical system includes a pressurized gas source, an aspiration chamber fluidly coupled to the pressurized gas source and containing a fluid disposed therein, a first valve fluidly coupled to the pressurized gas source and the aspiration chamber, a second valve fluidly coupled to the pressurized gas source and the aspiration chamber, an accumulator fluidly coupled to the pressurized gas source and the aspiration chamber between the first valve and the second valve, a pressure transducer fluidly coupled to the accumulator, and a computer electrically coupled to the first valve, the second valve, the accumulator, and the pressure transducer. The apparatus creates a reflux pressure pulse in the aspiration chamber by the computer maintaining the first valve in an open state and the second valve in a closed state, allowing pressurized gas to flow from the pressurized gas source through the first valve to form a pre-charge reflux pressure in the accumulator, closing the first valve, opening the second valve to discharge the pre-charge reflux pressure into the aspiration chamber, and re-closing the second valve. In another aspect of the present invention, the above-described apparatus creates a steady state reflux pressure in the aspiration chamber by the computer maintaining the first valve and the second valve in an open state, the pressure transducer determining an actual pressure within the accumulator and providing a first signal corresponding to the actual pressure to the computer, the computer comparing the actual pressure to a desired pressure within the accumulator, and the computer providing a second signal to adjust the proportional valve in response to the comparison of the actual pressure to the desired pressure.

### BRIEF DESCRIPTION OF THE DRAWINGS

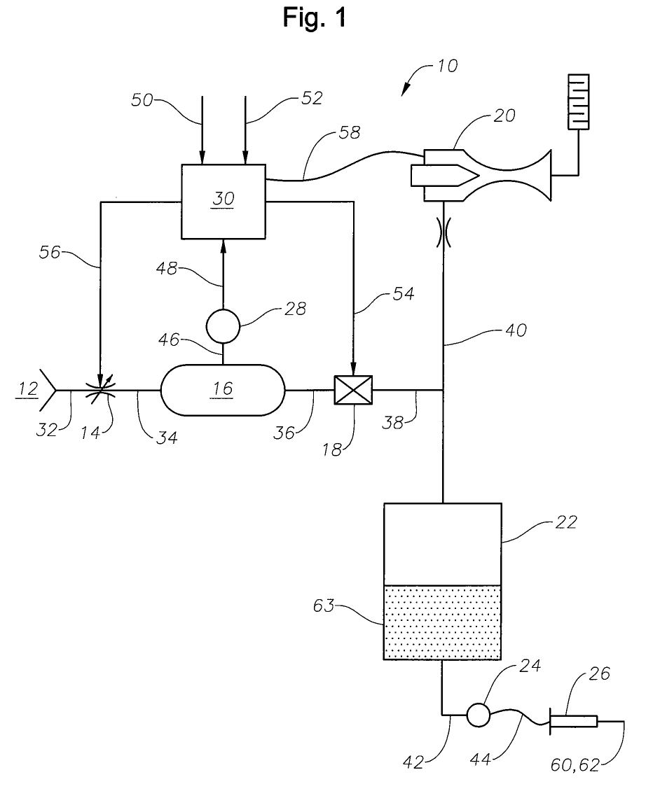

For a more complete understanding of the present invention, and for further objects and advantages thereof, reference is made to the following description taken in conjunction with the accompanying drawing, in which FIG. 1 is a schematic diagram illustrating an aspiration circuit of a microsurgical system.

### DETAILED DESCRIPTION OF THE PREFERRED EMBODIMENTS

The preferred embodiment of the present invention and its advantages is best understood by referring to FIG. 1 of the drawings. Microsurgical system 10 preferably includes a pressurized gas source 12, a proportional valve 14, an accumulator 16, an isolation valve 18, a vacuum generator 20, an aspiration chamber 22, an aspiration port 24, a surgical device 26, a pressure transducer 28, and a computer or microprocessor 30. The various components of system 10 are fluidly coupled via fluid lines 32, 34, 36, 38, 40, 42, 44, and 46. The various components of system 10 are electrically coupled via interfaces 48, 50, 52, 54, 56, and 58. Valve 14 is preferably a proportional solenoid valve. Accumulator 16 preferably has a volume of about 15 cc. Valve 18 is preferably an “on/off” solenoid valve. Vacuum generator 20 may be any suitable device for generating vacuum but is preferably a vacuum chip or a venturi chip that generates vacuum. Surgical device 26 may be any surgical device that aspirates tissue but is preferably an ophthalmic surgical device such as a phacoemulsification probe, a vitrectomy probe, or an aspiration probe. Surgical device 26 has a tip 60 with a port 62 that is fluidly coupled to fluid line 44. Pressure transducer 28 may be any suitable device for directly or indirectly measuring pressure and vacuum. Microprocessor 30 is capable of implementing feedback control, and preferably PID control.

The aspiration circuit of FIG. 1 enables improved control of reflux in microsurgical system 10. During normal operation of surgical device 26, fluid and/or tissue 63 are aspirated from port 62 into aspiration chamber 22 via vacuum supplied by vacuum generator 20. However, during a reflux operation of microsurgical system 10, microprocessor 30 sends a signal via interface 58 to turn off vacuum generator 20.

A user may input a setpoint for the desired pressure in accumulator 16 via interface 50. A user may also input whether a steady state reflux pressure or a pulsed reflux pressure is desired via interface 52. Alternatively, microprocessor 30 may provide a pre-defined reflux pressure profile for accumulator 16. Pressure transducer 46 measures the actual pressure within accumulator 16 and provides a corresponding signal to microprocessor 30 via interface 48. Microprocessor 30 compares the signal provided by pressure transducer 46 to the currently desired pressure for accumulator 16 and then adjusts proportional valve 14 via a signal over interface 56 so as to keep the measured reflux pressure of accumulator 16 at or near the desired reflux pressure.

When a steady state reflux pressure is commanded, microcontroller 30 maintains isolation valve 18 in an open position via a signal over interface 54. Microcontroller 30 then controls the reflux pressure within accumulator 16, aspiration chamber 22, and port 62 of surgical device 26 as described above. Since aspiration port 24 is located at the bottom of aspiration chamber 22, aspiration chamber 22 functions as a reservoir to provide sustained reflux, if necessary.

When a pulsed reflux pressure is commanded, microprocessor 30 momentarily closes isolation valve 18. Microprocessor 30 regulates the actual pressure within accumulator 16 as described above to create a “pre-charge” reflux pressure. Microprocessor 30 then closes proportional valve 14, opens isolation valve 18 to discharge the pre-charge reflux pressure in accumulator 16, and then re-closes isolation valve 18. In this manner, microprocessor 30 generates a pressure pulse that travels to aspiration chamber 22 and port 62 of surgical device 26. Such a pressure pulse is fully repeatable and programmable based upon the pre-defined reflux pressure profile stored in microprocessor 30.

Accumulator 16 also functions as a safety device. Once proportional valve 14 is closed, the maximum reflux pressure delivered to aspiration chamber 22 and port 62 is limited by the volume of accumulator 16 and the pre-charge reflux pressure.

The present invention is illustrated herein by example, and various modifications may be made by a person of ordinary skill in the art. For example, while the present invention is described above relative to reflux control in an ophthalmic microsurgical system, it is also applicable to other microsurgical systems.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A method of controlling reflux in a microsurgical system, comprising:
providing a pressurized gas source, an aspiration chamber fluidly coupled to said pressurized gas source and containing a fluid disposed therein, a first valve fluidly coupled to said pressurized gas source and said aspiration chamber, a second valve fluidly coupled to said pressurized gas source and said aspiration chamber, an accumulator fluidly coupled to said pressurized gas source and said aspiration chamber between said first valve and said second valve, a pressure transducer fluidly coupled to said accumulator, and a computer electrically coupled to said first valve, said second valve, and said pressure transducer; and
creating a reflux pressure pulse in said aspiration chamber by using said computer to maintain said first valve in an open state and said second valve in a closed state, to allow pressurized gas to flow from said pressurized gas source through said first valve to form a pre-charge reflux pressure in said accumulator, to close said first valve, to open said second valve to discharge said pre-charge reflux pressure into said aspiration chamber, and to re-close said second valve.

2. The method of claim 1 wherein said first valve is a proportional valve.

3. The method of claim 2 wherein said formation of said pre-charge reflux pressure in said creating step further comprises:
using said pressure transducer to determine an actual pressure within said accumulator and to provide a first signal corresponding to said actual pressure to said computer;
using said computer to compare said actual pressure to a desired pressure within said accumulator and to provide a second signal to adjust said proportional valve in response to said comparison of said actual pressure to said desired pressure.

4. The method of claim 1 further comprising repeating said creating step to create multiple ones of said reflux pressure pulses in a repetitive manner.

5. The method of claim 4 wherein said repeating step is performed according to a pre-defined profile in said computer.

6. The method of claim 1 further comprising the step of providing a surgical device having a tip with an open port fluidly coupled to said aspiration chamber.

7. A method of controlling reflux in a microsurgical system, comprising:
providing a pressurized gas source, an aspiration chamber fluidly coupled to said pressurized gas source and containing a fluid disposed therein, a proportional valve fluidly coupled to said pressurized gas source and said aspiration chamber, a second valve fluidly coupled to said pressurized gas source and said aspiration chamber, an accumulator fluidly coupled to said pressurized gas source and said aspiration chamber between said proportional valve and said second valve, a pressure transducer fluidly coupled to said accumulator; and a computer electrically coupled to said proportional valve, said second valve, and said pressure transducer;
creating a steady state reflux pressure in said aspiration chamber by:
using said computer to maintain said proportional valve and said second valve in an open state;
using said pressure transducer to determine an actual pressure within said accumulator and to provide a first signal corresponding to said actual pressure to said computer; and
using said computer to compare said actual pressure to a desired pressure within said accumulator and to provide a second signal to adjust said proportional valve in response to said comparison of said actual pressure to said desired pressure.

8. The method of claim 7 further comprising the step of providing a surgical device having a tip with an open port fluidly coupled to said aspiration chamber.
